(19) United States
(12) Patent Application Publication
(10) Pub. No.: US 2014/0120190 A1
(43) Pub. Date: May 1, 2014

(54) METHODS OF PREPARING PRICKLY-ASH PEEL EXTRACTS AND COSMETIC COMPOSITIONS INCORPORATING PRICKLY-ASH PEEL EXTRACTS

(71) Applicants: Dong-Qing WEI, Jiangsu (CN); Yu-Kun MA, Jiangsu (CN); Zhong-Dong QIAO, Jiangsu (CN)

(72) Inventors: Dong-Qing WEI, Jiangsu (CN); Yu-Kun MA, Jiangsu (CN); Zhong-Dong QIAO, Jiangsu (CN)

(21) Appl. No.: 14/059,289

(22) Filed: Oct. 21, 2013

(30) Foreign Application Priority Data
Oct. 20, 2012 (CN) 2012-10442824.5
Dec. 12, 2012 (CN) 2012-10595384.7
Mar. 14, 2013 (CN) 2013-10097498.3
Apr. 25, 2013 (CN) 2013-10168995.8

(51) Int. Cl. A61K 8/97; A61K 8/42; A61Q 19/08

(52) U.S. Cl. CPC A61K 8/97; A61Q 19/08; A61K 8/42
USPC 424/771

(57) ABSTRACT

Methods of preparing an extract of Prickly-ash peel, including pulverizing the peels of Chinese Prickly-ash berries to produce powdered Prickly-ash peel; extracting the powdered Prickly-ash peel using an organic solvent to yield an organic extract solution; and removing the organic solvent under reduced pressure to yield a residue that is an extract of Prickly-ash peel. The resulting Prickly-ash peel extracts include the active anti-aging ingredient gx-50, having the formula

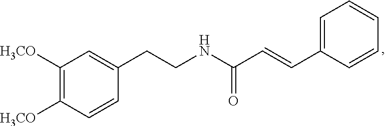 [SMILES: COc1ccc(CCNC(=O)/C=C/c2ccccc2)cc1OC]

which may be dispersed in a liquid or semi-solid physiologically and cosmetically acceptable vehicles or carriers to form topical anti-aging cosmetics.

### BACKGROUND

The skin is the single largest organ in the human body, and represents the first line of defense against the environment. As human beings age, however, the skin also ages, thinning and losing elasticity. Aging therefore affects overall appearance, but may also increase susceptibility to various skin diseases.

Several mechanisms have been proposed to explain the effects of aging on the human body, including free radical initiator, cumulative mitochondrial damage, and regulation of gene expression. With respect to the regulation of gene expression, it has been observed that the activity of the gene for NF-κB (Nuclear Factor κB) increases proportionally with age. The protein complex NF-κB is a transcriptional regulator that plays a central role in responses to inflammatory signaling.

Natural materials have proven to be a rich source of substances that possess anti-aging activity. These natural substances can be obtained from their natural sources much more readily than from chemical synthesis, or the use of biotechnology. In addition, natural products are much more likely to be accepted and trusted by consumers. Cosmetics that contain mainly herbs and other natural ingredients are typically favored by consumers, as they are perceived as possessing less toxicity and therefore higher safety. Such natural compositions have become part of the mainstream of the development of cosmetics.

Counteracting the effects of aging on skin is an area of active interest for both the health and cosmetics industries. For the reasons recited above, the development of anti-aging compositions containing natural active ingredients has become an active area of research and development.

### SUMMARY

The present invention includes a method of preparing an extract of Prickly-ash peel, the method including pulverizing at least the peels of Chinese Prickly-ash berries to produce powdered Prickly-ash peel; extracting the powdered Prickly-ash peel using an organic solvent to yield an organic extract solution; and removing the organic solvent under reduced pressure to yield a residue that is an extract of Prickly-ash peel.

The present invention additionally includes a method of preparing an extract of Prickly-ash peel that includes pulverizing at least the peels of Chinese Prickly-ash berries to produce powdered Prickly-ash peel; extracting the powdered Prickly-ash peel using methylene chloride, ethanol, or ethyl acetate to yield an organic extract solution; washing the organic extract solution with an alkaline aqueous solution; drying the washed organic extract solution using a drying agent; and removing the organic solvent under reduced pressure to yield a residue that is an extract of Prickly-ash peel.

The present invention yet additionally includes a topical anti-aging cosmetic that includes a prickly-ash peel extract that in turn includes an active ingredient gx-50 having the formula

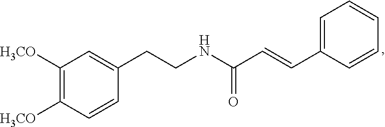 [SMILES: COc1ccc(CCNC(=O)/C=C/c2ccccc2)cc1OC]

such that the prickly-ash peel extract is dispersed in a liquid or semi-solid physiologically and cosmetically acceptable vehicle or carrier.

The present invention yet additionally includes a method of preparing a topical anti-aging cosmetic, that includes preparing an extract of Chinese Prickly-ash Peel; and dispersing the extract of Chinese Prickly-ash Peel in an emulsion that is a liquid or semi-solid physiologically and cosmetically acceptable vehicle or carrier.

### BRIEF DESCRIPTION OF THE DRAWINGS

FIG. 1 is a flowchart depicting a method of preparing an extract of Prickly-ash peel, according to embodiment of the present invention.

FIG. 2 is a flowchart depicting an alternative method of preparing an extract of Prickly-ash peel, according to another embodiment of the present invention.

FIG. 3 is a flowchart depicting a method of preparing a topical anti-aging cosmetic, according to another embodiment of the present invention.

### DETAILED DESCRIPTION

The Chinese Prickly-ash (Z. simulans or Z. bungeanum) is a spreading shrub or small tree that bears reddish brown berries. The husk, or peel, of the Prickly-ash berry is also known as Sichuan pepper, and is used in traditional Chinese cuisine.

The peel of the Chinese Prickly-ash has been found to contain a variety of small molecules, including the small molecule gx-50. Gx-50 has the following chemical structure:

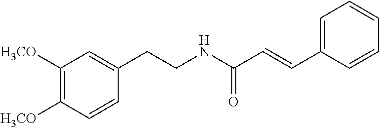 [SMILES: COc1ccc(CCNC(=O)/C=C/c2ccccc2)cc1OC]

The small molecule gx-50 effectively blocks the expression of the NF-κB gene by blocking the α-7 acetylcholine receptor, and thereby helps prevent the loss of collagen and elastin in skin at the tissue level. Prickly-ash peel extract has demonstrated a replenishing effect that compensates for massive losses of elastin and collagen, repairs hair follicles, inhibits the production of malondialdehyde (MDA) at the cellular level, and reduces senile plaques. Animal experiments have shown that after treating 20-month-old mice with Prickly-ash peel extracts for two weeks, both gene expression profiles and tissue signatures illustrate that the skin of the mice has been restored to a state equivalent to that of a younger mouse.

In addition to possessing an antioxidant effect, like traditional anti-aging ingredients such as superoxide dismutase (SOD), Vitamin C, and other antioxidants, gx-50 additionally acts as a regulator for NF-κB gene expression, and therefore possesses an enhanced potential to fundamentally slow the aging process. At the same time, the molecular weight of gx-50 is so small that gx-50 is more easily absorbed via the skin.

Based on the outstanding performance of Prickly-ash peel extracts for anti-aging, Prickly-ash peel extracts possess tremendous potential as active components for anti-aging formulations, such as cosmetics, which could in turn represent very high commercial values. An efficient and cost-effective method for obtaining Prickly-ash peel extract would significantly facilitate the development and manufacture of such anti-aging formulations.

### Preparation of Prickly-Ash Peel Extracts

As used herein, an extract is a composition prepared by removing a desired portion of a raw material. Such extraction is typically accomplished using an aqueous or organic solvent in which the desired portion is at least somewhat soluble. The resulting solution is a liquid extract.

Prickly-ash peel extract is derived from a raw material that includes the peels, or husks, of the berries of the Chinese Prickly-ash. The extract may be prepared from Prickly-ash peels (wherein the seeds of the berry have been removed), or may be prepared from the entire berry. The Prickly-ash peel may be used without further processing, but is typically at least partially crushed or ground before extraction to increase the surface area of the Prickly-ash peel particles, and increase extraction efficiency. In one embodiment, the Prickly-ash peel is crushed into a powder. Any means of grinding or milling that can reduce the Prickly-ash peel to smaller particles is a suitable method for the purposes of the invention, including hand grinding mills, powered grinding mills, and industrial grinding mills, such as ball mills, gristmills, hammer mills, disk mills, edge mills, and the like.

Where the extract is prepared using an organic solvent, the solvent should be sufficiently polar to extract the desired materials from the Prickly-ash peel (for example, gx-50). Polar organic solvents such as dichloromethane, ethanol, and ethyl acetate, among others, may be particularly useful for this purpose.

The extraction of the Prickly-ash peel may be performed using any method that brings the Prickly-ash peel into contact with the selected organic solvent in such a manner that at least a portion of the desired component(s) of the peel is dissolved in the solvent and removed from the peel. Conventional extraction methodologies include, for example, washing, immersion, percolation, decoction, reflux extraction, and continuous extraction (such as Soxhlet extraction). In one embodiment of the invention, the Prickly-ash peel is extracted using a continuous extraction method. Although solvent extraction may be performed at temperatures ranging from room temperature to the boiling point of the solvent selected for the extraction, typically the continuous extraction is performed at elevated temperatures, and preferably with the solvent under reflux, such as when using a Soxhlet extractor.

The extraction process creates a solution of the organic solvent that may include a number of natural materials removed from the Prickly-ash peel, including desirable components such as gx-50. The extract may also include any number of undesirable components, such as for example organic acids. If desired, the organic acids may be removed from the extract solution by washing the organic solution with an aqueous solution of base. The particular base used may be a relatively weak base, or a stronger base. In one embodiment of the invention, the organic extract solution is washed with a potassium carbonate (K₂CO₃) solution, a sodium hydroxide (NaOH) solution, or a potassium hydroxide (KOH) solution. Any water remaining in the washed organic solution can be removed by stirring the solution over anhydrous sodium sulfate (Na₂SO₄), then removing the drying agent by suction filtration.

The resulting organic phase corresponds to a partially purified solution of gx-50. The extract solution may be concentrated and/or purified further before use. For example, the solvent may be removed from the extract solution, leaving a solid, semi-solid, or oily residue. This resulting concentrated or dried extract may optionally be redissolved in the same or a different solvent. Alternatively, or in addition, the resulting concentrate may be further purified, for example by additional extractions, filtration, partial crystallization, chromatography, and the like. In a preferred embodiment, the solvent is removed from the washed organic extract solution under reduced pressure to yield an extract that is rich in gx-50.

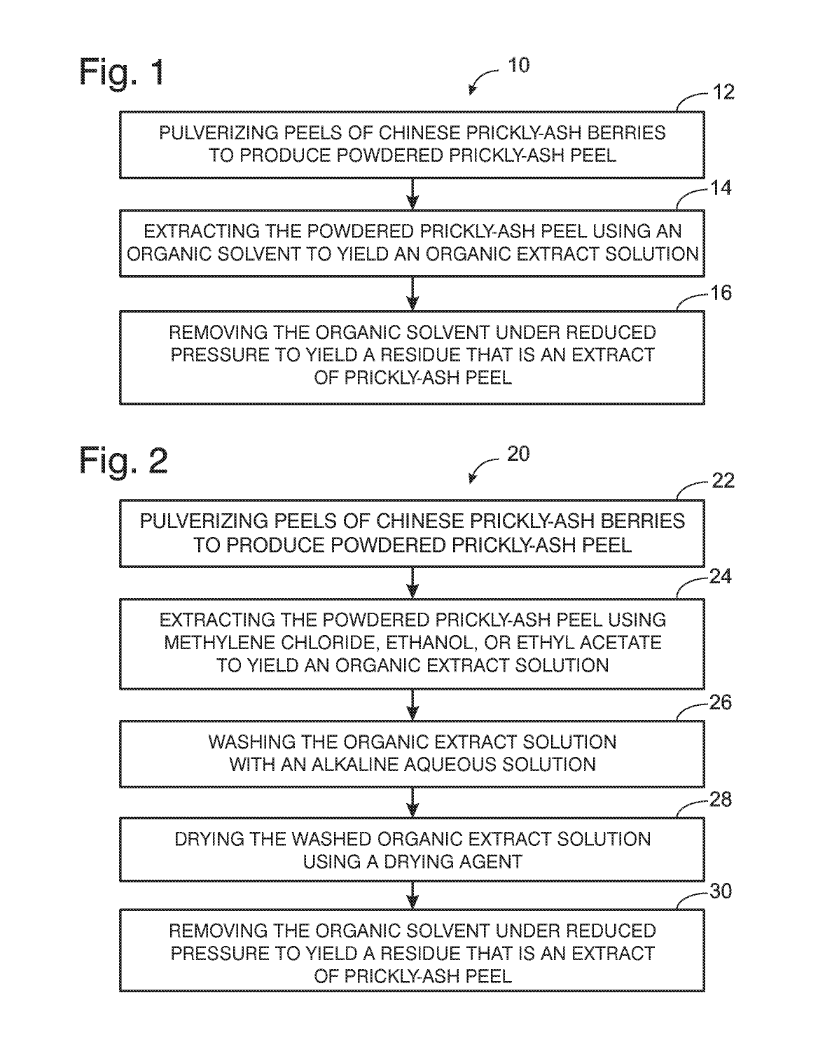

An extract of Prickly-ash peel may be prepared using the representative method depicted in flowchart 10 of FIG. 1, which includes pulverizing at least peels of Chinese Prickly-ash berries to produce powdered Prickly-ash peel at 12; extracting the powdered Prickly-ash peel using an organic solvent to yield an organic extract solution at 14; and removing the organic solvent under reduced pressure to yield a residue that is an extract of Prickly-ash peel at 16.

An alternative preparation for Prickly-ash peel extract is shown in flowchart 20 of FIG. 2, which includes pulverizing at least peels of Chinese Prickly-ash berries to produce powdered Prickly-ash peel at 22; extracting the powdered Prickly-ash peel using methylene chloride, ethanol, or ethyl acetate to yield an organic extract solution at 24; washing the organic extract solution with an alkaline aqueous solution at 26; drying the washed organic extract solution using a drying agent at 28; and removing the organic solvent under reduced pressure to yield a residue that is an extract of Prickly-ash peel 30.

Typically, the Prickly-ash peel extract includes at least 0.1% gx-50 by weight. In an alternative embodiment, the Prickly-ash peel extract includes least 0.3% gx-50 by weight. In a preferred embodiment, the Prickly-ash peel extract includes at least 1.0% gx-50 by weight.

### Anti-Aging Cosmetic Compositions

The present invention provides compositions for anti-aging cosmetics having gx-50 as their principle active ingredient. The cosmetic compositions may be prepared in accordance with the methods and techniques described below. The resulting compositions exhibit anti-aging, moisturizing, and generally nourishing effects on human skin.

More particularly, the present invention provides topical anti-aging cosmetic compositions that comprise a prickly-ash peel extract, where the prickly-ash peel extract includes an active ingredient gx-50 having the formula

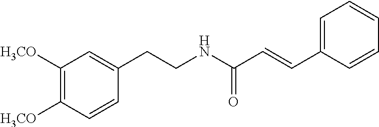 [SMILES: COc1ccc(CCNC(=O)/C=C/c2ccccc2)cc1OC]

The Prickly-ash peel extract, and therefore the gx-50, is typically dispersed in a liquid or semi-solid physiologically and cosmetically acceptable vehicle or carrier. As used herein, a physiologically and cosmetically acceptable vehicle or carrier suitable vehicle may act variously as solvent, diluent or dispersant for the constituents of the composition, and allows for the uniform application of the constituents to the surface of the skin at an appropriate dilution. It will be apparent to the skilled artisan that the range of possible vehicles is very broad. In general, compositions according to this invention may comprise water as a vehicle, and/or at least one physiologically and cosmetically acceptable vehicle other than water.

In addition to the active ingredient gx-50, the cosmetic formulations of the present invention may include any number of additional components, including for example thickening agents, moisturizing agents, dispersants, surfactants, preservatives, fragrances, and coloring agents.

The cosmetic formulations of the present invention may include polyhydric alcohol, which in turn may include 1,2-propanediol, glycerol, 1,3-butanediol, 1,2-pentanediol, dipropylene glycol, or any combination thereof.

The cosmetic formulations of the invention may include one or more moisturizing agents, or humectants. A moisturizing agent is any component that retains or otherwise preserves the moisture of the composition. Moisturizing agents may include one or more of various polyols, sodium lactate, urea, sorbitol, pyrrolidone carboxylic acid (PCA), gelatins, hyaluronic acid, vitamins, and proteins, among others.

The cosmetic formulations of the invention may include one or more thickening agents. A thickening agent is any component that confers a thicker consistency on the composition. The thickening agent may include one or more of cocamidopropylamine oxide (CAPO), cellulose, cellulose gum, carboxymethyl hydroxyethyl cellulose, cetyl hydroxyethyl cellulose, ethyl cellulose, hydroxyethyl cellulose, hydroxypropyl cellulose, hydroxypropylmethyl cellulose, methyl cellulose, carboxymethyl cellulose, acrylate/C10-C30 alkyl acrylate crosspolymers, acrylate/hexadecyl ethoxy (20) itaconate copolymers, acrylic ester/hexadecyl ethoxy (20) methacrylate copolymers, acrylic ester/tetradecyl ethoxy (25) acrylic acid ester copolymer, acrylate/stearyl ethoxy (20) itaconate copolymers, acrylate ester/stearyl ethoxy (20) methacrylate copolymers, acrylate/stearyl ethoxy (50) acrylic acid ester copolymers, acrylate/VA crosslinked polymers, PAA (polyacrylic acid), sodium acrylate/vinyl acetate, isodecyl crosslinked polymer, carbomer (polyacrylic acid) and its sodium salt, alginic acid and its ammonium, calcium, and potassium salts, pectin, sodium hyaluronate, guar gum, cationic guar gum, hydroxypropyl guar gum, tragacanth gum, carrageenan and its calcium and sodium salts, xanthan gum, sclerotium gum, magnesium aluminum silicate, sodium magnesium silicate, PVM/MA decadiene cross-linked polymer (polyvinyl methyl ether/methyl acrylate crosslinked with decadiene polymer), and PVP (polyvinyl pyrrolidone).

The cosmetic formulations of the invention may include one or more preservatives, which may be selected to preserve one or more desirable qualities of the cosmetic and/or extend its useful shelf-life. Such preservatives may include one or more of imidazolidinyl urea, isothiazolin-ketone, paraben esters, and quaternary ammonium salt-15, among others.

### Anti-Aging Cosmetic Compositions

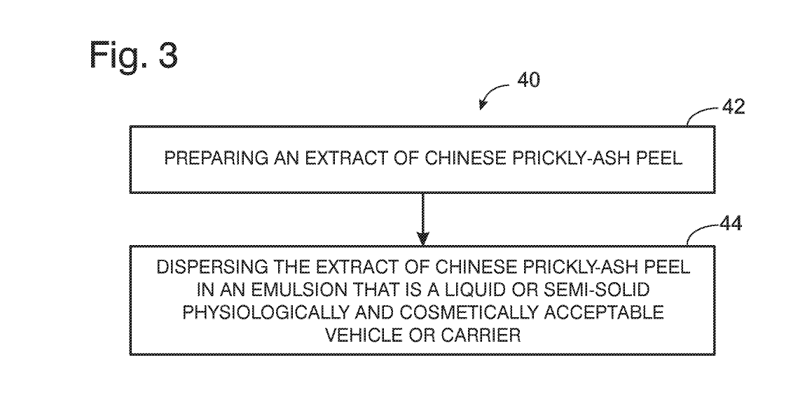

The present invention includes a method of preparing a topical anti-aging cosmetic, as depicted by flowchart 40 of FIG. 3, including preparing an extract of Chinese Prickly-ash peel at 42; and dispersing the extract of Chinese Prickly-ash Peel in an emulsion that is a liquid or semi-solid physiologically and cosmetically acceptable vehicle or carrier at 44.

The compositions of the present invention may have a formulation consistent with the following ingredients and amounts.

Component A

| Ingredient | Wt. Percent |
|---|---|
| Prickly-ash extract (gx-50) | 0.1-10.0 |
| Silicone oil | 0-1.8 |
| Squalane | 0-1.0 |
| Isododecane | 0-0.7 |
| Laurocapram (AZONE) | 0-0.8 |
| Vitamin A palmitate | 0.1-1.0 |
| Vitamin E | 0.1-0.8 |
| Borage oil | 3.0-10.0 |
| Jojoba oil | 0-4.0 |
| Mink oil | 1.0-3.0 |
| BHT | 0.02-0.06 |
| Stearyl alcohol/Cetyl alcohol | 1.0-2.0 |
| Stearic acid | 2.0-7.0 |
| Polyhydric alcohol | 3.0-5.5 |
| Isopropyl myristate | 0.5-2.0 |
| Six polyglycerol monostearate | 1.0-2.0 |

Component B

| Ingredient | Wt. Percent |
|---|---|
| Trimethylol diallyl ether | 1.0-2.0 |
| Moisturizing agent | 2.0-5.0 |
| EDTA | 0.02-0.08 |
| Water | 40-85 |

Component C

| Ingredient | Wt. Percent |
|---|---|
| Thickening agent | 0.2-0.8 |
| Preservative | 0.2-0.6 |

Component D

| Ingredient | Wt. Percent |
|---|---|
| Silk peptide | 0.5-3.0 |
| Perfume | As appropriate |

The above components may be combined to form a topical anti-aging cosmetic composition according to the following procedure:

Prepare Component A by combining the desired ingredients and heating the mixture to approximately 80-85° C. until completely dissolved.

Prepare Component B by combining the desired ingredients and heating the mixture to approximately 80-90° C.

Add Component B to Component A in two portions. First, add approximately ⅓ of Component B to Component A and stir rapidly for 3-5 minutes. Then, slowly add the remaining portion of Component B. Keeping the mixture homogeneous, maintain the temperature at 80-85° C. for 6-10 minutes. Slowly cool the mixture, with stirring, to 55-60° over a period of 1.2-2.0 hours.

Add Component C to the mixture, stirring with moderate intensity to maintain homogeneity for 3-5 minutes, then slowly cool the combined mixture to 35-37° C.

Add Component D to the mixture, then adjust the pH to 5-7 using NaOH or KOH. Maintain stirring for 20-30 minutes to obtain a uniform white stable product.

The following examples describe selected aspects and embodiments of the present invention. These examples are included for illustration and are not intended to limit or define the entire scope of the invention.

### EXAMPLES

### Example 1

Crushed Sichuan Dahongpao Prickly-ash Peel powder is weighed, and 100 g of the powder is added to a filter paper thimble and the thimble is placed into a Soxhlet extraction apparatus. The Soxhlet apparatus is fitted with a round-bottomed flask that is charged with dichloromethane, and the solvent is heated to reflux. The extraction is continued at reflux for 12-24 hours. After cooling, the extracts are washed three times with aqueous NaOH, then dried with anhydrous Na₂SO₄ which is removed by filtration. The solvent is then removed under reduced pressure to yield 5.23 g of a creamy deep red residue that is bitter and spicy. The residue contains 0.71% gx-50, as confirmed by High-Pressure Liquid Chromatography (HPLC) using a 150×15 mm C18 chromatography column and a mobile phase of methanol:water:0.1% acetic acid, detecting at 278 nm.

### Example 2

Crushed Hebei She County Prickly-ash Peel powder is weighed, and 100 g of the powder is added to a filter paper thimble and the thimble is placed into a Soxhlet extraction apparatus. The Soxhlet apparatus is fitted with a round-bottomed flask that is charged with dichloromethane, and the solvent is heated to reflux. The extraction is continued at reflux for 12-24 hours. After cooling, the extracts are washed three times with aqueous NaOH, then dried with anhydrous Na₂SO₄ which is removed by filtration. The solvent is then removed under reduced pressure to yield 11.03 g of an orange sticky liquid residue. The residue contains 1.21% gx-50, as confirmed by High-Pressure Liquid Chromatography (HPLC) using a 150×15 mm C18 chromatography column and a mobile phase of methanol:water:0.1% acetic acid, detecting at 278 nm.

### Example 3

Crushed Sichuan Dahongpao Prickly-ash Berry powder is weighed, and 100 g of the powder is added to a filter paper thimble and the thimble is placed into a Soxhlet extraction apparatus. The Soxhlet apparatus is fitted with a round-bottomed flask that is charged with dichloromethane, and the solvent is heated to reflux. The extraction is continued at reflux for 12-24 hours. After cooling, the extracts are washed three times with aqueous NaOH, then dried with anhydrous Na₂SO₄ which is removed by filtration. The solvent is then removed under reduced pressure to yield 9.83 g of an orange sticky liquid residue. The residue contains 0.14% gx-50, as confirmed by High-Pressure Liquid Chromatography (HPLC) using a 150×15 mm C18 chromatography column and a mobile phase of methanol:water:0.1% acetic acid, detecting at 278 nm.

### Example 4

Crushed Sichuan Dahongpao Prickly-ash Peel powder is weighed, and 100 g of the powder is added to a filter paper thimble and the thimble is placed into a Soxhlet extraction apparatus. The Soxhlet apparatus is fitted with a round-bottomed flask that is charged with dichloromethane, and the solvent is heated to reflux. The extraction is continued at reflux for 12-24 hours. After cooling, the extracts are washed three times with aqueous NaOH, then dried with anhydrous Na₂SO₄ which is removed by filtration. The solvent is then removed under reduced pressure to yield 14.5 g of a brownish-black semi-solid residue. The residue contains 2.42% gx-50, as confirmed by High-Pressure Liquid Chromatography (HPLC) using a 150×15 mm C18 chromatography column and a mobile phase of methanol:water:0.1% acetic acid, detecting at 278 nm.

### Example 5

Crushed Sichuan Dahongpao Prickly-ash Peel powder is weighed, and 100 g of the powder is added to a 500 mL round-bottomed flask. The powder is boiled in 200 mL, 180 mL, and 150 mL water, respectively, for 2-4 hours and then dried. The resulting residue is crushed and then extracted with dichloromethane for 12-24 hours. The dichloromethane extract is washed with aqueous NaOH three times, dried over anhydrous Na₂SO₄, and then filtered. The solvent is then removed under reduced pressure to yield 3.90 g of a creamy orange residue. The residue contains 1.46% gx-50, as confirmed by High-Pressure Liquid Chromatography (HPLC) using a 150×15 mm C18 chromatography column and a mobile phase of methanol:water:0.1% acetic acid, detecting at 278 nm.

### Example 6

Crushed Sichuan Dahongpao Prickly-ash Peel powder is weighed, and 100 g of the powder is added to a filter paper thimble and the thimble is placed into a Soxhlet extraction apparatus. The Soxhlet apparatus is fitted with a round-bottomed flask that is charged with ethanol, and the solvent is heated to reflux. The extraction is continued at reflux for 12-24 hours, and then the organic solvent is removed from the extract under reduced pressure. The resulting residue is dissolved in dichloromethane, and the solution is washed three times with aqueous NaOH, then dried with anhydrous Na₂SO₄ which is removed by filtration. The solvent is then removed under reduced pressure to yield 10.23 g of a creamy deep red residue that is bitter and spicy. The residue contains 0.32% gx-50, as confirmed by High-Pressure Liquid Chromatography (HPLC) using a 150×15 mm C18 chromatography column and a mobile phase of methanol:water:0.1% acetic acid, detecting at 278 nm.

### Example 7

Crushed Sichuan Dahongpao Prickly-ash Peel powder is weighed, and 100 g of the powder is added to a filter paper thimble and the thimble is placed into a Soxhlet extraction apparatus. The Soxhlet apparatus is fitted with a round-bottomed flask that is charged with ethyl acetate, and the solvent is heated to reflux. The extraction is continued at reflux for 12-24 hours, the extract is cooled, and the solution is washed three times with aqueous NaOH, then dried with anhydrous Na₂SO₄ which is removed by filtration. The solvent is then removed under reduced pressure to yield 8.36 g of a creamy deep red residue that is bitter and spicy. The residue contains 0.40% gx-50, as confirmed by High-Pressure Liquid Chromatography (HPLC) using a 150×15 mm C18 chromatography column and a mobile phase of methanol:water:0.1% acetic acid, detecting at 278 nm.

### Example 8

Prickly-ash peel extracts, as prepared according to Examples 1-7, may be incorporated as an active component of an anti-aging cosmetic composition. An exemplary anti-aging cosmetic may have the following ingredients in the following amounts:

| Component | Amount (wt %) |
|---|---|
| Glycerol Monostearate | 1.5 |
| Octadecyl Alcohol | 3 |
| Stearic Acid | 10 |
| PCA-Na (Pyrrolidone Carboxylic Acid-Na) | 10 |
| KOH | 0.5 |
| Methylparaben | As appropriate |
| Essence (fragrance) | As appropriate |
| Prickly-ash Peel extract | 1.5 |
| Water | 73.5 |

The glycerol monostearate, octadecyl alcohol and stearic acid are heated together to 80-90° C. and stirred evenly to obtain an oil phase. The PCA-Na and KOH are added to the water and the solution is heated to 80-90° C. to obtain an aqueous phase. The aqueous phase is then slowly added to the oil phase with vigorous stirring, and the resulting emulsion is permitted to cool slowly. The Prickly-ash peel extract, essence, and methylparaben are dissolved in a minimum amount of ethanol and the solution is added to the emulsion to yield a creamy white composition.

### Example 9

An alternative and exemplary anti-aging cosmetic incorporating Prickly-ash peel extract as prepared according to Examples 1-7.

Component 9A

| Ingredient | Wt. Percent |
|---|---|
| Prickly-ash extract (gx-50) | 1.4 |
| Silicone Oil | 0.7 |
| Squalane | 0.9 |
| Isododecane | 0.5 |
| Laurocapram (AZONE) | 0.5 |
| Vitamin A palmitate | 0.4 |
| Vitamin E | 0.4 |
| Borage oil | 3.6 |
| Jojoba oil | 2.0 |
| Mink oil | 1.5 |
| BHT | 0.04 |
| Stearyl alcohol/Cetyl alcohol | 1.0 |
| Stearic acid | 2.5 |
| 1,3-Butanediol | 3.6 |
| Isopropyl myristate | 1.4 |
| Six polyglycerol monostearate | 1.4 |

Component 9B

| Ingredient | Wt. Percent |
|---|---|
| Trimethylol diallyl ether | 1.6 |
| Pyrrolidone carboxylic acid-Na | 3.6 |
| EDTA | 0.02 |
| Water | Balance |

Component 9C

| Ingredient | Wt. Percent |
|---|---|
| Cocamidopropylamine Oxide (CAPO) | 0.2-0.8 |
| Preservative | 0.3 |

Component 9D

| Ingredient | Wt. Percent |
|---|---|
| Silk peptide | 1.8 |
| Perfume | As appropriate |

Component 9A by combining the desired ingredients and heating the mixture to approximately 80-85° C. until completely dissolved. Component 9B is prepared by combining the desired ingredients and heating the mixture to approximately 80-90° C. Component 9B is added to Component 9A in two portions: Approximately ⅓ of Component 9B is added to Component 9A with rapid stirring for 3-5 minutes. The remaining portion of Component 9B is then added. Keeping the mixture homogeneous, the temperature of the mixture is maintained at a temperature of 80-85° C. for 6-10 minutes. The mixture is then slowly cooled, with stirring, to 55-60° over a period of 1.2-2.0 hours.

Component 9C is then added to the mixture, stirring with moderate intensity to maintain homogeneity for 3-5 minutes. The resulting mixture is then slowly cooled to 35-37° C.

Component 9D is added to the combined mixture, and the pH is adjusted to 5-7 using NaOH or KOH. Stirring is maintained for 20-30 minutes to obtain a uniform white stable product.

### Example 10

An alternative and exemplary anti-aging cosmetic incorporating Prickly-ash peel extract as prepared according to Examples 1-7.

Component 10A

| Ingredient | Wt. Percent |
|---|---|
| Prickly-ash extract (gx-50) | 2.0 |
| Silicone Oil | 0.5 |
| Squalane | 0.7 |
| Isododecane | 0.3 |
| Vitamin A palmitate | 0.3 |
| Vitamin E | 0.3 |
| Borage oil | 3.0 |
| Mink oil | 1.0 |
| BHT | 0.05 |
| Stearyl alcohol/Cetyl alcohol | 1.0 |
| Stearic acid | 4.5 |
| 1,3-Butanediol | 5.0 |
| Isopropyl myristate | 1.0 |
| Six polyglycerol monostearate | 1.5 |

Component 10B

| Ingredient | Wt. Percent |
|---|---|
| Trimethylol diallyl ether | 1.5 |
| Pyrrolidone carboxylic acid-Na | 2.0 |
| EDTA | 0.02 |
| Water | Balance |

Component 10C

| Ingredient | Wt. Percent |
|---|---|
| Cocamidopropylamine Oxide (CAPO) | 0.3 |
| Preservative | 0.4 |

Component 10D

| Ingredient | Wt. Percent |
|---|---|
| Silk peptide | 1.8 |
| Perfume | As appropriate |

The cosmetic composition is prepared using a procedure analogous to the procedure of Example 9, above.

Although the present invention has been shown and described with reference to the foregoing operational principles and preferred embodiments, it will be apparent to those skilled in the art that various changes in form and detail may be made without departing from the spirit and scope of the invention. The present invention is intended to embrace all such alternatives, modifications and variances that fall within the scope of the appended claims.

The present invention should be considered to include all novel and non-obvious combinations and subcombinations of the various elements, features, functions and/or properties disclosed herein. Similarly, where the claims recite “a” or “a first” element or the equivalent thereof, such claims should be understood to include incorporation of one or more such elements, neither requiring nor excluding two or more such elements.

Inventions embodied in various combinations and subcombinations of features, functions, elements, and/or properties may be claimed through presentation of new claims in a related application. Such new claims, whether they are directed to a different invention or directed to the same invention, whether different, broader, narrower or equal in scope to the original claims, are also regarded as included within the subject matter of the inventions of the present disclosure.

What is claimed is:

1. A method of preparing an extract of Prickly-ash peel, comprising:
pulverizing at least peels of Chinese Prickly-ash berries to produce powdered Prickly-ash peel;
extracting the powdered Prickly-ash peel using an organic solvent to yield an organic extract solution; and
removing the organic solvent under reduced pressure to yield a residue that is an extract of Prickly-ash peel.

2. The method of claim 1, wherein the organic solvent is methylene chloride, ethanol, ethyl acetate, or any mixture thereof.

3. The method of claim 1, further comprising washing the organic extract solution with an alkaline aqueous solution.

4. The method of claim 3, wherein the alkaline aqueous solution includes at least one of K₂CO₃, NaOH, or KOH.

5. The method of claim 3, further comprising drying the washed organic extract solution using a drying agent.

6. The method of claim 5, wherein the drying agent is aqueous brine, anhydrous CaCl₂, anhydrous CaSO₄, anhydrous MgSO₄, anhydrous K₂CO₃, or anhydrous Na₂SO₄.

7. The method of claim 1, wherein the extract is enriched in gx-50 with respect to the peels of Chinese Prickly-ash berries.

8. The method of claim 1, wherein the extract includes at least 1.0% gx-50 by weight.

9. The method of claim 1, wherein extracting the powdered Prickly-ash peel using an organic solvent includes extracting the powdered Prickly-ash peel via dipping, percolation, decoction, reflux extraction, or continuous extraction.

10. The method of claim 1, wherein extracting the powdered Prickly-ash peel using an organic solvent includes extracting the Prickly-ash peel via reflux extraction or continuous extraction.

11. A method of preparing an extract of Prickly-ash peel, comprising:
pulverizing at least peels of Chinese Prickly-ash berries to produce powdered Prickly-ash peel;
extracting the powdered Prickly-ash peel using methylene chloride, ethanol, or ethyl acetate to yield an organic extract solution;
washing the organic extract solution with an alkaline aqueous solution;
drying the washed organic extract solution using a drying agent;
removing the organic solvent under reduced pressure to yield a residue that is an extract of Prickly-ash peel.

12. The method of claim 13, wherein the resulting extract of Prickly-ash peel contains at least 1.0% by weight of a compound gx-50 having the structure:

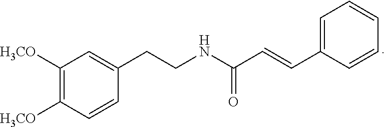 [SMILES: COc1ccc(CCNC(=O)/C=C/c2ccccc2)cc1OC]

13. A topical anti-aging cosmetic comprising:
a prickly-ash peel extract including an active ingredient gx-50 having the formula 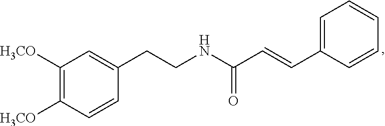 [SMILES: COc1ccc(CCNC(=O)/C=C/c2ccccc2)cc1OC]
wherein the prickly-ash peel extract is dispersed in a liquid or semi-solid physiologically and cosmetically acceptable vehicle or carrier.

14. The topical anti-aging cosmetic of claim 13, wherein the prickly-ash peel extract is present in the cosmetic in a concentration of about 0.1 to 10.0% by weight.

15. The topical anti-aging cosmetic of claim 13, further comprising one or more additional components selected from thickening agents, moisturizing agents, dispersants, surfactants, preservatives, fragrances, and coloring agents.

16. The topical anti-aging cosmetic of claim 15, further comprising one or more of silicone oil, squalane, isododecane, laurocapram, Vitamin A palmitate, Vitamin E, borage oil, jojoba oil, mink oil, BHT, stearyl alcohol/cetyl alcohol, stearic acid, polyhydric alcohol, isopropyl myristate, six polyglycerol monostearate, trimethylol diallyl ether, EDTA, silk peptide,

17. The topical anti-aging cosmetic of claim 15, wherein the moisturizing agent includes one or more of polyols, sodium lactate, urea, sorbitol, pyrrolidone carboxylic acid, gelatin, hyaluronic acid, vitamins, and proteins.

18. The topical anti-aging cosmetic of claim 15, wherein the thickening agent includes one or more of cocamidopropylamine oxide (CAPO), cellulose, cellulose gum, carboxymethyl hydroxyethyl cellulose, cetyl hydroxyethyl cellulose, ethyl cellulose, hydroxyethyl cellulose, hydroxypropyl cellulose, hydroxypropylmethyl cellulose, methyl cellulose, carboxymethyl cellulose, acrylate/C10-C30 alkyl acrylate crosspolymers, acrylate/hexadecyl ethoxy (20) itaconate copolymers, acrylic ester/hexadecyl ethoxy (20) methacrylate copolymers, acrylic ester/tetradecyl ethoxy (25) acrylic acid ester copolymer, acrylate/stearyl ethoxy (20) itaconate copolymers, acrylate ester/stearyl ethoxy (20) methacrylate copolymers, acrylate/stearyl ethoxy (50) acrylic acid ester copolymers, acrylate/VA crosslinked polymers, PAA (polyacrylic acid), sodium acrylate/vinyl acetate, isodecyl crosslinked polymer, carbomer (polyacrylic acid) and its sodium salt, alginic acid and its ammonium, calcium, and potassium salts, pectin, sodium hyaluronate, guar gum, cationic guar gum, hydroxypropyl guar gum, tragacanth gum, carrageenan and its calcium and sodium salts, xanthan gum, sclerotium gum, magnesium aluminum silicate, sodium magnesium silicate, PVM/MA decadiene cross-linked polymer (polyvinyl methyl ether/methyl acrylate crosslinked with decadiene polymer), and PVP (polyvinyl pyrrolidone).

19. The topical anti-aging cosmetic of claim 16, wherein polyhydric alcohol includes one or more of 1,2-propanediol, glycerol, 1,3-butanediol, 1,2-pentanediol, and dipropylene glycol.

20. The topical anti-aging cosmetic of claim 16, having the following composition:

| Ingredient | Wt. Percent |
|---|---|
| Prickly-ash extract (gx-50) | 0.1-10.0 |
| Silicone oil | 0-1.8 |
| Squalane | 0-1.0 |
| Isododecane | 0-0.7 |
| Laurocapram (AZONE) | 0-0.8 |
| Vitamin A palmitate | 0.1-1.0 |
| Vitamin E | 0.1-0.8 |
| Borage oil | 3.0-10.0 |
| Jojoba oil | 0-4.0 |
| Mink oil | 1.0-3.0 |
| BHT | 0.02-0.06 |
| Stearyl alcohol/Cetyl alcohol | 1.0-2.0 |
| Stearic acid | 2.0-7.0 |
| Polyhydric alcohol | 3.0-5.5 |
| Isopropyl myristate | 0.5-2.0 |
| Six polyglycerol monostearate | 1.0-2.0 |
| Trimethylol diallyl ether | 1.0-2.0 |
| Moisturizing agent | 2.0-5.0 |
| EDTA | 0.02-0.08 |
| Water | 40-85 |
| Thickening agent | 0.2-0.8 |
| Preservative | 0.2-0.6 |
| Silk peptide | 0.5-3.0 |
| Perfume | As appropriate |

21. A method of preparing a topical anti-aging cosmetic, comprising:
preparing an extract of Chinese Prickly-ash Peel; and
dispersing the extract of Chinese Prickly-ash Peel in an emulsion that is a liquid or semi-solid physiologically and cosmetically acceptable vehicle or carrier.

22. The method of claim 21, further comprising:
adding the extract of Chinese Prickly-ash Peel to an oil phase; and
forming an emulsion of the oil phase and an aqueous phase.

23. The method of claim 22, wherein each of the oily phase and aqueous phase further include one or more thickening agents, moisturizing agents, preservatives, and fragrances.

24. The method of claim 21, further comprising:
forming an oil phase that includes glycerol monostearate, octadecyl alcohol, and stearic acid;
heating the oil phase;
forming an aqueous phase that includes pyrrolidone carboxylic acid-Na and KOH;
forming an emulsion by adding the aqueous phase to the oil phase; and
dispersing the Chinese Prickly-ash Peel extract into the emulsion.
